(19) United States
(12) Patent Application Publication
(10) Pub. No.: US 2003/0138797 A1
(43) Pub. Date: Jul. 24, 2003

(54) Nucleic acid-based compounds

(75) Inventors: Radhakrishnan P. Iyer, Shrewsbury, MA (US); Yi Jin, Carlsbad, CA (US); Arlene Roland, Castries (FR)

Correspondence Address: SEED INTELLECTUAL PROPERTY LAW GROUP PLLC, 701 FIFTH AVE, SUITE 6300, SEATTLE, WA, 98104-7092, US

(73) Assignee: Micrologix Biotech Inc., Vancouver (CA)

(21) Appl. No.: 10/183,631

(22) Filed: Jun. 28, 2002

Related U.S. Application Data
(60) Provisional application No. 60/301,991, filed on Jun. 29, 2001.
(60) Provisional application No. 60/302,875, filed on Jul. 2, 2001.

(51) Int. Cl. C12Q 1/68; C07H 21/02; C08G 63/48; C08G 63/91; C07H 21/04

(52) U.S. Cl. 435/6; 525/54.2; 536/23.1

(57) ABSTRACT

The present invention relates to novel phosphoramidate compounds and libraries containing such compounds, and methods for preparing such compounds. Compounds of the invention will be useful in a variety of applications, e.g. as a nucleoside or oligonucleotide therapeutic agent, or for diagnostic or analytical applications, e.g. as a capture probe in a hybridization assay.

This application claims the benefit of U.S. Provisional Patent Application No. 60/301,991, filed Jun. 29, 2001 and U.S. Provisional Patent Application No. 60/302,875, filed Jul. 2, 2001.

### BACKGROUND OF THE INVENTION

1. Field of the Invention

The present invention relates to novel phosphoramidate compounds and libraries containing such compounds, and methods for preparing such compounds. Compounds of the invention will be useful in a variety of applications, e.g. as a nucleoside or oligonucleotide therapeutic agent, or for diagnostic or analytical applications, e.g. as a capture probe in a hybridization assay.

2. Background

A variety of nucleic-acid based compounds have been investigated for therapeutic applications as well as for a range of analytical methods, including as capture probes in hybridization assays. See, for instance, U.S. Pat. No. 5,736,316.

The important initial step in the development of therapeutic agents is the discovery of compounds that bind to a protein, enzyme or receptor of interest. Through careful structure/activity work of resulting active compounds, one arrives at a lead compound for further development into a clinical candidate. That traditional process of drug discovery can be a long and arduous endeavor. Often it takes 10 to 15 years before a new drug makes it into the marketplace.

Certain more recent approaches to the discovery of therapeutics have been developed. In one more modem approach, large libraries of diverse compounds are synthesized and subjected to high throughput screening against a particular molecular target implicated in a disease.

### SUMMARY OF THE INVENTION

The invention provides nucleotide-based compounds and libraries that comprise phosphoramidate linkages.

Compounds of the invention are useful as therapeutic agents, particularly antiviral agents. In particular, compounds of the invention will have use against hepatisis viruses. See, for instance, the results set forth in Example 3, which follows.

The invention also provides phosphoramidate compounds that are covalently linked, particularly via an unsaturated linker group. Preferred linker groups include aromatic or conjugated systems that comprise hydroxy and alkylhydroxy substituents that can facilitate electron transfer and thereby decoupling of the compound from a solid support.

The invention also provides phosphoramidate compounds covalently linked to a solid structure, particularly a reaction body, for use in a wide variety of diagnostic and other analytical applications. In particular, a phosphoramidate oligonucleotide can be covalently linked to a substrate surface, such as a glass reaction surface, and used as a capture probe in analysis of genetic material. In this aspect, the invention include microarray platforms that comprise a phosphoramidate oligonucleotide of the invention, preferably covalently linked to a reaction space of the microarray.

The invention further provides methods for synthesis of nucleotide-based compounds and new libraries of such compounds.

Preferred synthetic methods of the invention include methods for parallel assembly of phosphoramidate libraries.

Particularly preferred synthetic methods of the invention include methods for preparing a plurality of phosphoramidate compounds, comprising contacting a plurality of nucleoside reagents with one or more amines to thereby provide a plurality of phosphoramidate compounds. Suitably, a plurality of distinct amine reagents (e.g. primary or secondary acylic, alicyclic or aromatic amines) are reacted with the nucleoside reagents to provide a library that contains distinct phosphoramidate compounds. The reaction may be suitably solid phase (e.g. nucleoside reagents are covalently bound to a solid support) or solution phase. The plurality of phosphoramidate compounds are prepared in a single reaction sequence. See, for instance, Scheme 1 below. A wide variety of libraries may be prepared, such as libraries that contain 10, 20, 50, 100, 200, 300, 400, 500, 600, 700, 800, 900 or 1000 or more compounds.

Other aspects of the invention are discussed infra.

### DETAILED DESCRIPTION OF THE INVENTION

As discussed above, phosphoramidate compounds and libraries of such compounds are provided.

Preferred synthetic methods of the invention include the approach outlined in the following Scheme 1:

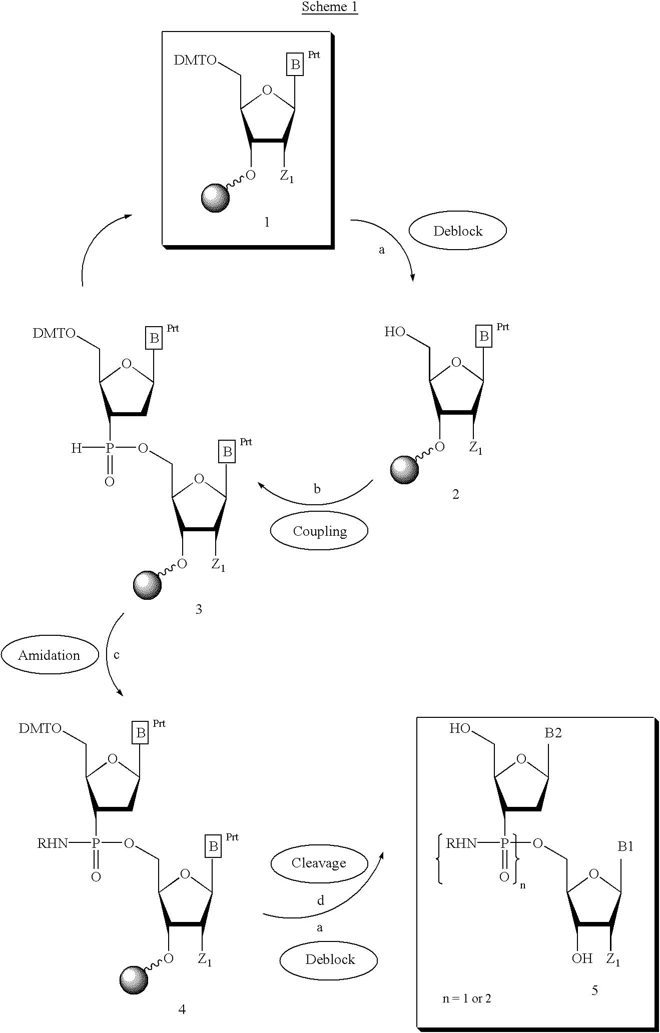

In Scheme 1, Z₁, is a suitable substituent, preferably R₃ or OR₃, and B and R₃ are as defined below for Formulas I, II, III and IV. As outlined in the above Scheme, support bound nucleoside 1 is suitably deprotected (e.g. under acetic conditions such as 3% dichloroacetic acid in CH₂Cl₂) to the deprotected bound nucleoside 2. Additional nucleoside units may be coupled to the bound unit to give 3, preferably in the presence of an activator such as adamantine carbonyl chloride. The phosphoramidate linkage 4 is then suitably generated by an amidation reaction, such as e.g. by reaction with a desired amine (such as the preferred amines set forth in Table 1 below) suitably in a solvent such as a 10% CCl₄ solution. The resulting phosphoramidate 5 can be decoupled from the solid support by, for example, treatment with a base such as, but not limited to, NH₄OH.

The above procedure can provide yields of from e.g. 10 to 95% and crude purities of 50 to 95%. Yields and/or purities can be enhanced by alternate reagents and/or conditions, particularly as may be determined for a specific amine reagent, which alternate reagent and/or conditions can be readily determined empirically.

More specifically, in the method outlined in Scheme 1, the requisite solid-support-bound dinucleoside H-phosphonates (5′-DMT-off) are suitably assembled on CPG-support using nucleoside H-phosphonates in conjunction with an activator such as 1-adamantane carbonyl chloride. Each support-bound H-phosphonate is treated with an amine, including a mixture of different amines to provide a library of distinct members, preferably with the amine in solution. The resulting phosphoramidates can be released from the solid-support suitably by treatment with base, such as aqueous NH₄OH, as mentioned above.

Improvement in yields of the library members can be accomplished, for example, by use of oxidative amidation conditions. In the case of non-hindered amines, amidation was performed using either pyridine/CCl₄, or triethylamine/CCl₄, the purity of the library members could be improved to 95% in an overall yield of 50% after purification. Reverse phase HPLC was effective for purification of library members having aromatic amines. Also noted is that reaction of dibenzyl amine resulted in partial debenzylation and formation of the corresponding monobenzylated product.

A variety of amines are preferably coupled to a nucleoside unit to provide a phosphoramidate linkage in accordance with the invention. For instance, acyclic primary, secondary and tertiary amines may be reacted with a nucleoside unit. Carbon alicyclic and heteroalicyclic amines and aromatic amines, including carbocyclic aryl-substituted amines and heteraromatic amines may be reacted and substituted onto a phosphoramidate group.

Preferred acyclic amines to react with a nucleoside unit to provide a phosphoramidate linkage include optionally substituted aminoalkyl groups and include those groups having one or more primary, secondary and/or tertiary amine groups, and from 1 to about 12 carbon atoms, more preferably 1 to about 8 carbon atoms, still more preferably 1, 2, 3, 4, 5 or 6 carbon atoms.

Suitable carbocyclic aryl amines and carbon alicyclic amines to react with a nucleoside unit to provide a phosphoramidate linkage suitably include one or more, typically one or more, exocyclic amine substituents, such as those discussed above. Typical carbocyclic aryl groups have one or more exocyclic amine groups and contain 1 to 3 separate or fused rings and from 6 to about 18 carbon ring atoms. Specifically preferred carbocyclic aryl groups include phenyl; naphthyl including 1-naphthyl and 2-naphthyl; biphenyl; phenanthryl; anthracyl; and acenaphthyl. Especially preferred carbocyclic aryl groups include optionally substituted phenyl with one or more amine substituents (such as anilines), optionally substituted naphthyl having one or more amine substituents, optionally substituted carbon alicyclic having 3 to about 30 ring carbons and 1-3 rings such as cyclopropyl, cyclobutyl, cyclopentyl, cyclohexyl, adamantly, norbornyl, all having one or more amine substituents. The amine substituents of the carbocyclic aryl and carbon alicyclic groups suitably may be one or more of the acyclic amines discussed above.

Suitable heteroaromatic and heteroalicyclic groups to react with a nucleoside unit to provide a phosphoramidate linkage suitably include an amine moiety, either as an exocyclic substituent, or as a ring member, and 1-3 N, O or S ring members and from 6 to about 18 total ring members, and 1-3 separate or fused rings. Specifically suitable heteroaromatic reagents to react with a nucleoside unit to provide a phosphoramidate linkage include optionally substituted pyridine, piperidine, pyrazine and pyrimidine, and the like. Suitable heteroalicyclic groups to react with a nucleoside unit to provide a phosphoramidate linkage include optionally substituted pyrrolidine, triazole, imidazole, indane, tetrahydrofuranyl, thienyl, tetrahydropyranyl, and the like.

Specifically preferred amines to react with a nucleoside unit to provide a phosphoramidate in accordance with the invention include those set forth in the following Table 1.

TABLE 1
Acyclic Amines

| | |
|---|---|
| | N₁ |
| 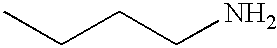 | |
| | N₂ |
| 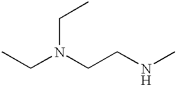 | |
| | N₃ |
| 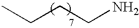 | |
| | N₄ |
| 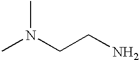 | |
| | N₅ |
| 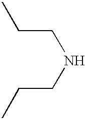 | |
| | N₆ |
| 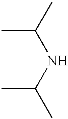 | |

Cyclic and Heterocyclic Amines

| | |
|---|---|
| | N₇ |
| 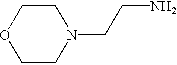 | |
| | N₈ |
| 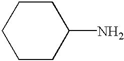 | |
| | N₉ |
| 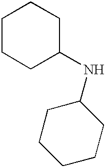 | |

| | |
|---|---|
| | N₁₀ |
| 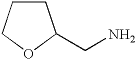 | |
| | N₁₁ |
| 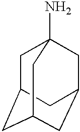 | |
| | N₁₂ |
| 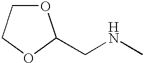 | |
| | N₁₃ |
| 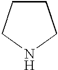 | |
| | N₁₄ |
| 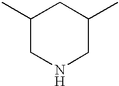 | |
| | N₁₅ |
| 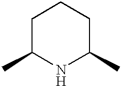 | |

Aromatic Amines

| | |
|---|---|
| | N₁₆ |
| 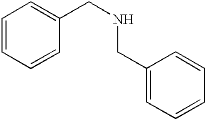 | |
| | N₁₇ |
| 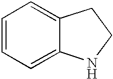 | |
| | N₁₈ |
| 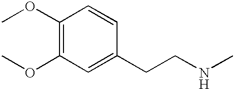 | |
| | N₁₉ |
| 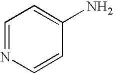 | |
| | N₂₀ |
| 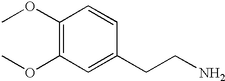 | |
| | N₂₁ |
| 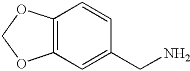 | |
| | N₂₂ |
| 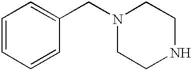 | |
| | N₂₃ |
| 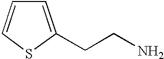 | |

Preferred phosphoramidate units of the invention include those of the following Formula I, II, III and IV:

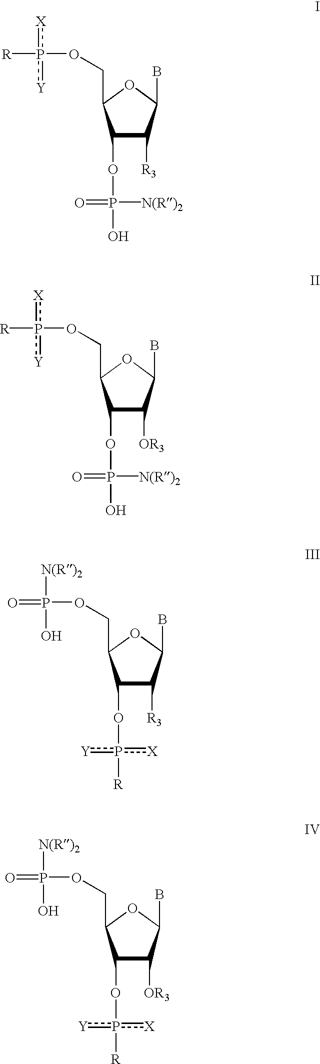

wherein in each of Formulae I, II, III and IV: X and Y are each independently O, S, Se, NR¹NR², CR¹CR², OR, SR, SeR, or an enzymatically reactive (particularly, cleavable) moiety such as an amide, ester and the like;

R, R¹, R² and R³ are independently hydrogen or a non-hydrogen group such as a hydrophobic group, e.g. a moiety having from 1 to about 18 carbon atoms, such as optionally substituted alkyl, optionally substituted alkenyl, optionally substituted alkynyl, optionally substituted aralkyl, optionally substituted cycloalkyl, optionally substituted cycloalkenyl, optinally substituted carbocyclic aryl, an optionally substituted mononucleotide, an optionally substituted polynucleotide, or an optionally substituted heteroaromatic or heteroalicyclic group preferably having from 1 to 3 separate or fused rings and 1 to 3 N, O or S atoms;

each R″ group is independently selected from the same group of substituents as defined for R, or to R″ moieties are taken together with the nitrogen to form a heteroalicyclic or heteroaromatic moiety with a nitrogen ring member;

B is a base, preferably optionally substituted adenine, optionally substituted thymidine, optionally substituted cytosine or optionally substituted guanine, preferably where the optional substituents are alkyl, carbocyclic aryl, or heteroaromatic or heteroalicyclic group preferably having from 1 to 3 separate or fused rings and 1 to 3 N, O or S atoms, or a heteroalicyclic structure that is covalently linked to the sugar ring; and pharmaceutically acceptable salts thereof.

Particularly preferred compounds of the invention include those of the following Formulae V and VI:

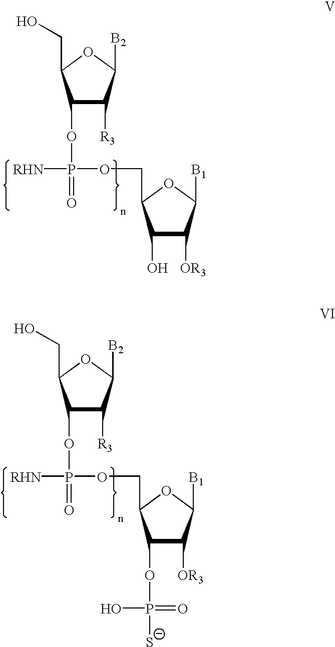

wherein in each of Formula V and VI:

each R and each R³ are each independently hydrogen or a non-hydrogen group, such as a hydrophobic group, eg. a moiety having from 1 to about 18 carbon atoms, such as optionally substituted alkyl, optionally substituted alkenyl, optionally substituted alkynyl, optionally substituted aralkyl, optionally substituted cycloalkyl, optionally substituted cycloalkenyl, optionally substituted carbocyclic aryl, an optionally substituted mononucleotide, an optionally substituted polynucleotide, or an optionally substituted heteroaromatic or heterolicyclic group preferably having from 1 to 3 separate or fused rings and 1 to 3 N, O or S atoms;

B₁, and B₂ are each the same or different and are each a base; and n is a positive integer, preferably 2 to about 100, more preferably 2, 3, 4, 5, 6, 7, 8, 9, 10 to about 20 to 25; and pharmaceutically acceptable salts of such compounds.

Preferred R and R³ groups of Formula V and VI include hydrogen, optionally substituted alkyl, optionally substituted alkenyl, optionally substituted alkynyl, optionally substituted aralkyl, optionally substituted cycloalkyl, optionally substituted cycloalkenyl, optionally substituted carbocyclic aryl, an optionally substituted mononucleotide, an optionally substituted polynucleotide, or an optionally substituted heteroaromatic or heteroalicyclic group preferably having from 1 to 3 separate or fused rings and 1 to 3 N, O or S atoms.

Preferred B₁, and B₂ groups of Formulae V and VI include optionally substituted adenine, optionally substituted thymidine, optionally substituted cytosine or an optionally substituted guanine.

Preferred R and R″ groups of compounds of the above Formulae I through VI include cyclic groups, particularly alicyclic groups that may comprise one or more single or polycyclic rings, particularly a bridged or fused ring structure, with 0, 1 or 2 endocyclic carbon-carbon double bonds. Additional preferred R and R″ groups include heteroalicyclic moieties, particularly heteroalicyclic. Particularly preferred R and R″ groups of a phosphoramidate group provide those acyclic, alicyclic and aromatic amines as discussed above.

Preferred compounds of the invention include those of the above formulae where the nucleoside is linked to the R group via a phosphorous group at the 5′ end. Such linkages could also be established via the 2′ or 3′ sites of the nucleoside. When R is a nucleoside, linkages can be via 5′ to 3′, 5′ to 5′, 3′ to 3′, 2′ to 5′ and 2′ to 2′, or any combination thereof, of the participating nucleosides.

The depicted sugar group of the above formulae may be natural or modified (e.g. synthetic) form, or in an open chain form (where one of the depicted ring bonds would not be present).

As mentioned above, X and Y of the above formulae may be an enzymatically reactive group, i.e. the group may be cleavable or otherwise reactive in vivo upon administration to a mammal, particularly a human. Preferred enzymatically reactive groups include e.g. amides (which may be cleaved in vivo with an amidase), esters (which may be cleaved in vivo with an esterase), and acetal and ketal groups.

Preferred compounds of the invention include those of the above formula where the nucleoside is linked to the R group via a phosphorous group at the 5′ end. Such linkages could also be established via the 2′ or 3′ sites of the nucleoside. When R is a nucleoside, linkages can be via 5′ to 3′, 5′ to 5′, 3′ to 3′, 240 to 5′ and 2′ to 2′, or any combination thereof, of the participating nucleosides.

As discussed, various substituents including R, R″, R¹, R², and R³ of the above formulae may be optionally substituted. A “substituted” R, R″, R¹, R², and R³ group or other substituent may be substituted by other than hydrogen at one or more available positions, typically 1 to 3 or 4 positions, by one or more suitable groups such as those disclosed herein. Suitable groups that may be present on a “substituted” R, R″, R¹, R2, and R3 group or other substituent include, for example, halogen such as fluoro, chloro, bromo and iodo; cyano; hydroxyl; nitro; azido; alkanoyl such as a C₁₋₆ alkanoyl group such as acyl and the like; carboxamido; alkyl groups, including those groups having 1 to about 12 carbon atoms, preferably 1, 2, 3, 4, 5, or 6 carbon atoms; alkenyl and alkynyl groups, including groups having one or more unsaturated linkages and from 2 to about 12 carbon atoms, preferably 2, 3, 4, 5 or 6 carbon atoms; alkoxy groups, including those having one or more oxygen linkages and from 1 to about 12 carbon atoms, preferably 1, 2, 3, 4, 5 or 6 carbon atoms; aryloxy such as phenoxy; alkylthio groups including those moieties having one or more thioether linkages and from 1 to about 12 carbon atoms, preferably 1, 2, 3, 4, 5 or 6 carbon atoms; alkylsulfinyl groups including those moieties having one or more sulfinyl linkages and from 1 to about 12 carbon atoms, preferably 1, 2, 3, 4, 5 or 6 carbon atoms; alkylsulfonyl groups including those moieties having one or more sulfonyl linkages and from 1 to about 12 carbon atoms, preferably 1, 2, 3, 4, 5 or 6 carbon atoms; aminoalkyl groups such as grous having one or more N atoms and from 1 to about 12 carbon atoms, preferably 1, 2, 3, 4, 5 or 6 carbon atoms; carbocyclic aryl having 6 or more carbons; aralkyl having 1 to 3 separate or fused rings and from 6 to about 18 carbon ring atoms, with benzyl being a preferred group; aralkoxy having 1 to 3 separate or fused rings and from 6 to about 18 carbon ring atoms, with 0-benzyl being a preferred group; or a heteroaromatic or heteroalicyclic group having 1 to 3 separate or fused rings with 3 to about 8 members per ring and one or more N, O or S atoms, e.g. coumarinyl, quinolinyl, pyridyl, pyrazinyl, pyrimidyl, furyl, pyrrolyl, thienyl, thiazolyl, oxazolyl, imidazolyl, indolyl, benzofuranyl, benzothiazolyl, tetrahydrofuranyl, tetrahydropyranyl, piperidinyl, morpholino and pyrrolidinyl.

Preferably, compounds of the invention will be present in enantiomerically enriched mixtures, i.e. where one enantiomer is present in a greater amount than other stereoisomer(s) of the compound, particularly where one enantiomer is present in amount of at least about 60 mole percent, relative to all steroisomers present of the compound; preferably where one enantiomer is present in an amount of at least about 70 or 80 mole percent, relative to all stereoisomers present of the compound; still more preferably where one enantiomer is present in amount of at least about 85, 90, 92, 95, 96, 97, 98 or 99 mole percent, relative to all stereoisomers present of the compound.

By “alkyl” in the present invention is meant straight or branched chain alkyl groups that preferably contain from 1 to about 18 carbon atoms, more preferably from 1 to about 12 carbon atoms and most preferably from 1 to about 6 carbon atoms. Specific examples of alkyl groups include, for example, methyl, ethyl, n-propyl, isopropyl, n-butyl, t-butyl, isobutyl, pentyl, hexyl, heptyl, octyl, nonyl, decyl, etc.

By “alkoxy” in the present invention is meant straight or branched chain alkyl groups that preferably contain from 1 to about 18 carbon atoms attached through at least one divalent oxygen atom, such as, for example, methoxy, ethoxy, propoxy, isopropoxy, n-butoxy, sec-butoxy, tert-butoxy, pentoxy, isopentoxy, neopentoxy, hexoxy, and 3-methylpentoxy.

“Alkenyl” means straight and branched hydrocarbon radicals preferably having from 2 to 18 carbon atoms and at least one double bond and includes ethenyl, propenyl, 1-but-3-enyl, 1-pent-3-enyl, 1-hex-5-enyl and the like.

“Alkynyl” means straight and branched hydrocarbon radicals preferably having from 2 to 18 carbon atoms and at least one triple bond and includes ethynyl, propynyl, butynyl, pentyn-2-yl and the like.

By the term “halogen” in the present invention is meant fluorine, bromine, chlorine, and iodine.

By “aryl” is meant an aromatic carbocyclic group having a single ring (e.g., phenyl), multiple rings (e.g., biphenyl), or multiple condensed rings in which at least one is aromatic, (e.g., 1,2,3,4-tetrahydronaphthyl, naphthyl). Also, in the above formulae, “aralkyl” groups include the above-listed alkyl groups substituted by a carbocyclic aryl group having 6 or more carbons, for example, phenyl, naphthyl, phenanthryl, anthracyl, etc.

By “heteroaryl” or “heteroaromatic” is meant one or more aromatic ring systems of 5-, 6-, or 7-membered rings which includes fused ring systems of 9-11 atoms containing at least one and up to four heteroatoms selected from nitrogen, oxygen, or sulfur. Such heteroaryl groups include, for example, thienyl, furanyl, thiazolyl, imidazolyl, (is)oxazolyl, pyridyl, pyrimidinyl, (iso)quinolinyl, napthyridinyl, benzimidazolyl, benzoxazolyl.

By “heteroalicyclic” is meant one or more carbocyclic ring systems of 4-, 5-, 6-, or 7-membered rings which includes fused ring systems of 9-11 atoms containing at least one and up to four heteroatoms selected from nitrogen, oxygen, or sulfur.

As used herein, the term “cycloalkyl” refers to saturated carbocyclic radicals having three to twelve carbon atoms. The cycloalkyl can be monocyclic, or a polycyclic fused system. In the above formulae, cycloalkyl groups preferably have from 3 to about 8 ring carbon atoms, e.g. cyclopropyl, cyclopentyl, cyclohexyl, cycloheptyl, e,4-methylenecyclohexane, adamantly, cyclopentylmethyl, cyclohexylmethyl, 1- or 2-cyclohexylethyl and 1-, 2- or 3-cyclohexylpropyl, etc.

In the above formulae, exemplary heteroaromatic and heteroalicyclic group include pyridyl, pyrazinyl, pyrimidyl, furyl, pyrrolyl, thienyl, thiazolyl, oxazolyl, imidazolyl, indolyl, benzothiazolyl, thtrahydrofuranyl, tetrahydropyranyl, piperidinyl, morpholino and pyrrolidinyl.

Mononucleotides of compounds of the invention include adenine, cytodine, guanosine and thymidine.

Polynucleotides of compounds of the invention preferably contain from about 1 to about 20 mononuculeotides, more preferably from 1 to about 10 mononuculeotides and still more preferably from 1 to about 5 nomonuculeotides. The polynucleotides are suitably constructed such that the 5′ group of one mononucleotide pentose ring is attached to the 3′ group of its neighbor in one direction via, for example, a phosphodiester or a phosphorthioate internucleotide linkage.

Sugar groups of compounds of the invention may be comprised of mono-, di-, oligo- or poly-saccharides wherein each monosaccharide unit comprises from 3 to about 8 carbons, preferably from 3 to about 6 carbons, containing polyhydroxy groups or polyhydroxy and amino groups. Non-limiting examples include glycerol, ribose, fructose, glucose, glucosamine, mannose, galactose, maltose, cellobiose, sucrose, starch, amylose, amylopectin, glycogen and cellulose. The hydroxyl and amino groups are present as free or protected groups containing e.g. hydrogens and/or halogens. Preferred protecting groups include acetonide, t-butoxy carbonyl groups, etc. Monosaccharide sugar groups may be of the L or D configuration and a cyclic monosaccharide unit may contain a 5 or 6 membered ring of the α or β conformation. Disaccharides may be comprised of two identical or two dissimilar monosaccharide units. Oligosaccharides may be comprised of from 2 to 10 monosaccharides and may be homopolymers, heteropolymers or cyclic polysugars. Polysaccharides may be homoglycans or heteroglycans and may be branched or unbranched polymeric chains. The di-, oligo- and poly-saccharides may be comprised of 1 4, 1 6 or a mixture of 1 6 linkages. The sugar moiety may be attached to the link group through any of the hydroxyl or amino groups of the carbohydrate.

As mentioned above, linker groups may be employed that are useful for linking a nucleic-acid based compound, particularly a phosphoramidate compound, to a solid support such as a glass or polymer substrate. The linker group preferably is aromatic or otherwise has multiple bonds that can facilitate electron transfer and is substituted by at least one hydroxy or amino group and at least one alkylamino or alkylhydroxy group, such as C₁₋₈-alkylamino or C₁₋₈-alkylhydroxy, particularly —CH₂NH₂ and —CH₂OH. The multiple bond moiety of the linker may be, for example, a single or fused ring compound such as phenyl, naphthyl and the like, or separate linked rings such as bi-phenyl that can enable electron transfer, or a non-aromatic conjugated system. For instance, suitable linkers include the following compounds A through F. In those structures A through F below, X and Y each represent a carbon or hetero atom such O, S or N, and the group M represent one or more non-hydrogen ring substituents such as halo, or a group as defined for R.

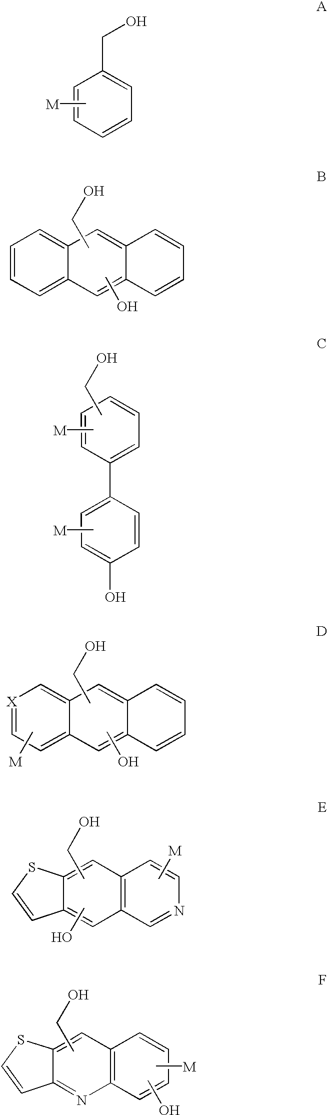

Compound libraries of the invention preferably will contain at least about 2,3,4 or 5 distinct compounds, more preferably at least about 10 distinct compounds, still more preferably at least about 20, 30, 40, 50, 60, 70, 80, 90 or 100 compounds, and may contain 200, 300, 400, 600, 700, 800, 900, or 1000 or more compounds.

Compounds of the invention will be useful for a variety or therapeutic application, including in methods of treatment against infections and diseases associated with a virus, particularly a hepadnavirus such as HBV. The invention thus includes methods of treatment of a mammal susceptible to (prophylactic treatment) or suffering from a disease associated with a virus, particularly a hepadnavirus, especially hepatitis B (HBV) virus. Methods of the invention generally include administration to a mammal, particularly a primate such as a human, in need of treatment a therapeutically effective amount of one or more compounds of the invention.

Compounds of the invention may be used as inhibitors of viral kinases, viral polymerases, and as disrupters of helicase-primase complexes with nucleic acids during viral replication.

Administration of compounds of the invention may be made by a variety of suitable routes including oral, topical (including transdermal, buccal or sublingual), nasal and parenteral (including intraperitoneal, subcutaneous, intravenous, intradermal or intramuscular injection) with oral or parenteral being generally preferred. It also will be appreciated that the preferred method of administration and dosage amount may vary with, for example, the condition and age of the recipient.

Compounds of the invention may be used in therapy in conjunction with other pharmaceutically active medicaments, such as another anti-viral agent, or an anti-cancer agent. Additionally, while one or more compounds of the invention may be administered alone, they also may be present as part of a pharmaceutical composition in mixture with conventional excipient, i.e., pharmaceutically acceptable organic or inorganic carrier substances suitable for parenteral, oral or other desired administration and which do not deleteriously react with the active compounds and are not deleterious to the recipient thereof. Suitable pharmaceutically acceptable carriers include but are not limited to water, salt solutions, alcohol, vegetable oils, polyethylene glycols, gelatin, lactose, amylose, magnesium stearate, talc, silicic acid, viscous paraffin, perfume oil, fatty acid monoglycerides and diglycerides, petroethral fatty cid esters, hydroxymethyl-cellulose, polyvinylpyrrolidone, etc. The pharmaceutical preparations can be sterilized and if desired mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously react with the active compounds.

For parental application, particularly suitable are solutions, preferably oily or aqueous solutions as well as suspensions, emulsions, or implants, including suppositories. Ampules are convenient unit dosages.

For enteral application, particularly suitable are tablets, dragees or capsules having talc and/or carbohydrate carrier binder or the like, the carrier preferably being lactose and/or corn starch and/or potato starch. A syrup, elixir or the like can be used wherein a sweetened vehicle is employed. Sustained release compositions can be formulated including those wherein the active component is protected with differentially degradable coatings, e.g., by microencapsulation, multiple coatings, etc.

Therapeutic compounds of the invention also may be incorporated into liposomes. The incorporation can be carried out according to known liposome preparation procedures, e.g. sonication and extrusion. Suitable conventional methods of liposome preparation are also disclosed in e.g. A. D. Bangham et al., J. Mol. Biol, 23:238-252 (1965); F. Olson et al., Biochim. Biophys. Acta, 557:9-23 (1979); F. Szoka et al., Proc. Nat. Acad. Sci., 75:4194-4198 (1978); S. Kim et al., Biochim. Biophys. Acta, 728:339-348 (1983); and Mayer et al., Biochim. Biophys. Acta, 858:161-168 (1986).

It will be appreciated that the actual preferred amounts of active compounds used in a given therapy will vary according to the specific compound being utilized, the particular compositions formulated, the mode of application, the particular site of administration, etc. Optimal administration rates for a given protocol of administration can be readily ascertained by those skilled in the art using conventional dosage determination tests.

As discussed above, compounds of the invention also may be used in diagnostic and other analytical methods, particularly in array platforms such as where a compound is covalently attached to the array platform such as a glass slide. The linked compound may be e.g. an oligonucleotide having from 2 to about 100 residues, more typically about 4 to about 10, 15, 20, 25, 30, 35, 40, 45, 50, 60, 70 or 80 residues. In use, a test sample, e.g. a patient's fluid sample (e.g. blood sample) for a diagnostic application, can be applied to the linked oligonucleotide on the reaction body (e.g. glass slide) and desired hybridization detected.

The disclosures in this application of all articles and references, including patents, are incorporated herein by reference.

The invention is illustrated further by the following examples which are not to be construed as limiting the invention in scope or spirit to the specific procedures described in them.

The starting materials and various intermediates may be obtained from commercial sources, prepared from commercially available organic compounds, or prepared using well known synthetic methods.

Representative examples of methods for preparing intermediates of the invention are set forth below.

### EXAMPLES

### Example 1

Library Assembly

Di- and tri-nucleoside phosphoramidates. The H-phosphonates were assembled on a controlled-pore-glass (CPG) support on a 10 μmol scale using standard H-phosphonate chemistry. The dry CPG-bound H-phosphonate was transferred to 5 mL RV tubes in a Quest 210™ Library Synthesizer and a solution of amine in CCl₄ (10%, 3 mL) was added. In the case of hindered amines, the reaction was performed in the presence of pyridine/CCl₄, or triethylamine/CCl₄, or collidine/CCl₄. The reaction mixture was agitated for 20-30 min. After washing, the CPG was transferred to 5 mL tube and treated with 28% aq NH₄OH (55° C., overnight). The suspension was cooled and centrifuged. The solution was evaporated to dryness in vacuo, residue dissolved in H₂O (5 mL), and extracted with ethyl acetate (2 mL). The purity of the resulting crude library members ranged from 85 to 95%.

The compounds were purified and desalted by passing through a C18-column (10×1 cm) (Buffer A: H₂O, Buffer B: 20% CH₃CN in H₂O) to give individual library members of 95-99% purity as determined by reversed-phase HPLC.

Post-column synthesis protocols for trinucleoside phosphoramidates were as described for dinucleoside phosphoramidates.

RP-HPLC analysis for crude products showed purity range of 50-80%. The crude product was purified by C18 column as before to give individual library members of up to 95% purity.

Chimeric trinucleotides (PS-PN and PN-PS). The requisite PS linkage in the trinucleotides was constructed using phosphoramidite chemistry (see Beaucage et al., Tetrahedron, 48: 2223 (1992)), in conjunction with 3H-1,2-benzodithiole-3-one-1,1-dioxide as the sulfurizing reagent. See Iyer et al., J. Am. Chem. Soc., 112:1253 (1990). The PN-linkage was incorporated using H-phosphonate chemistry.

For the 3 PS-PN library, the PS-linked dinucleotides were prepared on a synthesizer using standard phosphoramidite synthesis cycle (DMt-on). The CPG-column was dried and installed on another DNA synthesizer (BioSearch Model 8700) to establish the H-phosphonate linkage. The reverse synthetic sequence was employed for the PN-PS library. In both cases, the conversion of H-phosphonates to phosphoramidates was done on the Quest 210™ Synthesizer as previously described.

The crude compounds (50-75% pure) were purified on ion- exchange columns followed by desalting (C18 column) as before to give individual library members of up to 95% purity.

HPLC Analysis of Library Members:

RP-HPLC analysis of the libraries was performed on a Waters 600 system equipped with a photodiode-array UV detector 996, 717 autosampler, and Millennium® 2000 software, using a Radial-Pak® cartridge (8 mm I.D., 8NVC18). Mobile phase: Buffer A: 0.1 M NH₄OA_{c}; Buffer B: 20% A/80% CH₃CN, v/v: Gradient: 100% A, 0-3 min; 40% A, 40 min; 100% B, 49 min; 100% B. Product purity ranged from 85 to 95%.

Characterization of Library Members

Spectral characterization of representative library members was carried out on desalted and purified material. ³¹P NMR (D₂O, 85% H₃PO₄ external standard, ppm) 11-15 (PN linkage) and 56-58 (PS linkage). ¹H NMR analysis of selected compounds was consistent with the assigned structures. Additionally, the ES-MS of library members gave the expected molecular ions. Typical data are as follows.

3′AG-N₁₃. ³¹P NMR (D₂O), 12.18, 12.06 ppm. ES-MS: calcd for 633.2 (M); found: m/z 634.2 (M+H).

3′UA-N₁₈. ³¹P NMR (D₂O), 14.11, 13.74 ppm. ES-MS: Calcd. For 748.3; found: m/z, 749.1 (M+H).

Using the above methods, a 600-member library was prepared that include members shown in the following Table 2.

TABLE 2
Di- and tri-nucleoside phosphoramidate library

| | | | |
|---|---|---|---|
| 3′AA-Nx | 3′AG-Nx | 3′AA-Nx | 3′AG-Nx |
| 3′CA-Nx | 3′CG-Nx | 3′CA-Nx | 3′CG-Nx |
| 3′GA-Nx | 3′GG-Nx | 3′GA-Nx | 3′GG-Nx |
| 3′AC-Nx | 3′AT-Nx | 3′AC-Nx | 3′AT-Nx |
| 3′CC-Nx | 3′CT-Nx | 3′CC-Nx | 3′CT-Nx |
| 3′GC-Nx | 3′GT-Nx | 3′GC-Nx | 3′GT-Nx |
| 3′TC-Nx | 3′TT-Nx | 3′UC-Nx | 3′UT-Nx |

Nx corresponds to amines (see Table 1); A, C, G, T correspond to deoxyribonucleosides; A, C, G, U correspond to 2′-OMe ribonucleosides.

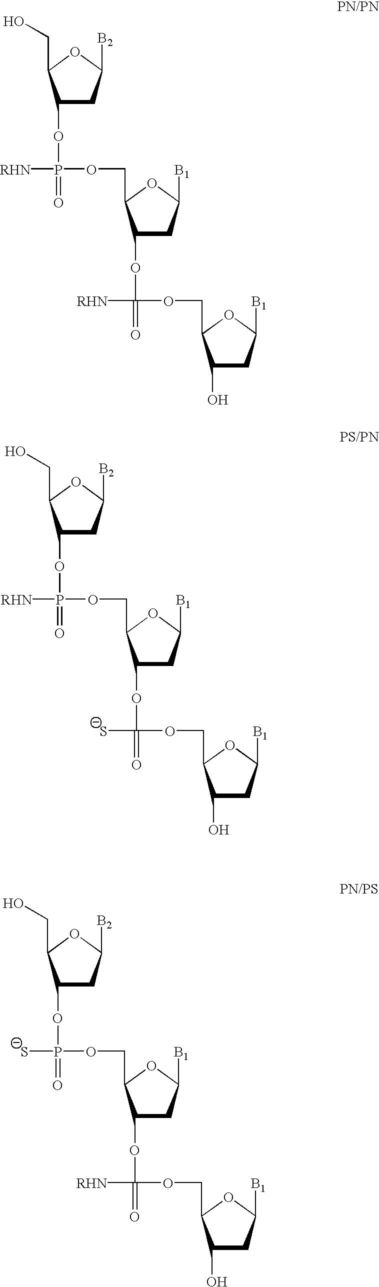

NHR=N₁-N₁₀, N₁₁, N₁₃, N₁₇, N₁₈, and N₂₀₋ N₂₃ (see Table 1).

### Example 2

Anti-HBV Activity

The anti-HBV activity, and cytotoxicity assays of the compounds were performed at 10 M concentration using HepG2-derived 2.2.15 cell lines according to published procedures using 3TC (IC₅₀-0.06 M) as the positive control. Korba et al., Antiviral Res. 1992, 20, 55. For the single-dose antiviral and toxicity analyses, confluent cultures 2.2.15 cells were maintained on 96-well flat-bottomed tissues culture plates in RPMI 1640 medium with 2% fetal bovine serum. Cultures were treated with nine consecutive daily doses of 10 M of the test compounds. Medium was changed daily with addition of fresh test compounds. Extracellular (virion) HBV DNA levels were measured 24 h after the last treatment.

For the multiple-dose analyses, two separate (replicate) plates were used for each antiviral drug treatment. A total of 3 cultures on each plate were treated with each of four serial 10-fold dilutions of antiviral agents (six cultures per dilution) for the antiviral assays.

Toxicity analyses for the multiple-dose treatments were performed on separate plates than those used for the antiviral assays. Cells for the toxicity analyses were cultured under conditions as used for the antiviral evaluations. Each compound or compound was tested at four concentrations, each in triplicate cultures. Uptake of neutral red dye was used to determine the relative level of toxicity 24 h following the last treatment. The absorbance of internalized dye at 510 nM (A₅₁₀) was used for the quantitative analysis. Representative antiviral data is as follows.

3′AG-N₁₃. IC₅₀2.1±0.2 M; CC₅₀>300 M; 3′UA-N₁₈: IC₅₀ 4.4±0.2 M: CC₅₀>300 M.

The invention and the manner and process of making and using it, are now described in such full, clear, concise and exact terms as to enable any person skilled in the art to which it pertains, to make and use the same. It is to be understood that the foregoing describes preferred embodiments of the present invention and that modifications may be made therein without departing from the spirit or scope of the present invention as set forth in the claims. To particularly point out and distinctly claim the subject matter regarded as invention, the following claims conclude this specification.

What is claimed is:

1. A compound comprising a structure of the following Formula I, II, III or IV covalently bound to a solid surface: 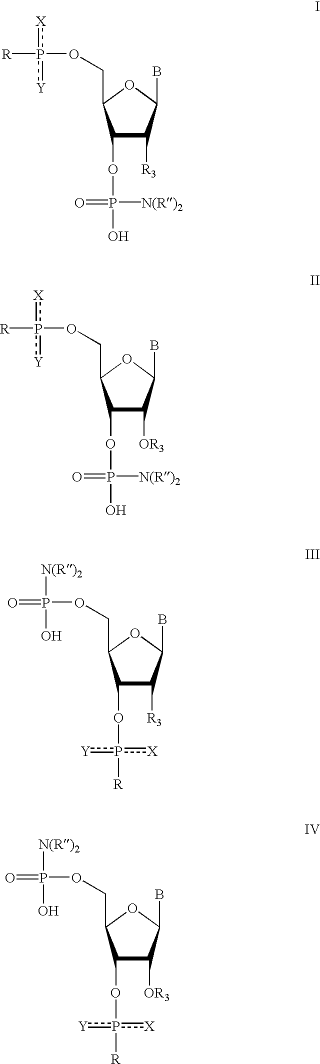
wherein in each of Formula I, II, III or IV: X and Y are each independently selected from the group consisting of O, S, Se, NR¹NR², CR¹CR², OR, SR and SeR, or one or both of X and Y are an enzymatically reactive moiety;
R is a hydrogen or non-hydrogen substituent;
each R″ is independently is hydrogen or a non-hydrogen substituent, or two R′ groups are taken together with the depicted nitrogen to form a ring;
R¹, R² and R³ are each independently selected from a group as defined by R;
B is base; and pharmaceutically acceptable salts thereof.

2. A compound of claim 1 wherein R, each R″, R¹, R² and R³ are each independently selected from the group of hydrogen, optionally substituted alkyl, optionally substituted alkenyl, optionally substituted alkynyl, optionally substituted aralkyl, optionally substituted cycloalkyl, optionally substituted cycloalkenyl, optionally substituted carbocyclic aryl, an optionally substituted mononucleotide, an optionally substituted polynucleotide, or an optionally substituted heteroaromatic or heteroalicyclic group preferably having from 1 to 3 separate or fused ring and 1 to 3 N, O or S atoms.

3. A compound of claim 1 wherein B is optionally substituted adenine, optionally substituted thymidine, optionally substituted cytosine or an optionally substituted guanine.

4. A compound of claims 1 wherein the support is a reaction body.

5. A compound of claim 1 wherein the substrate is a microarray substrate.

6. A compound of claim 1 wherein the substrate contains a plurality of oligonucleotides covalently linked to the substrate.

7. A compound of claim 1 wherein the compound is covalently bound to the substrate via an interposed linker.

8. A compound of claim 7 wherein the linker comprises two or more carbon-carbon unsaturated groups.

9. A compound of claim 7 wherein the linker comprises an aromatic group.

10. A compound of claim 7 wherein the linker has a hydroxy or amino substituent.

11. A compound of claim 7 wherein the linker has hydroxy and alkylhydroxy substituents.

12. A compound of claim 1 wherein the plurality of physphoramidate compounds are each independently selected from the following Formulae V and VI: 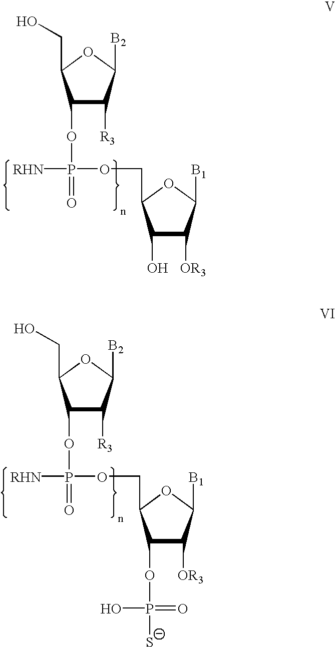
wherein in each of Formula V and VI:
each R and each R³ are each independently hydrogen or a non-hydrogen group;
B₁ and B₂ are each a base; and n is a positive integer; and pharmaceutically acceptable salts thereof.

13. A library of compounds having a structure of the following Formula I, II, III or IV: 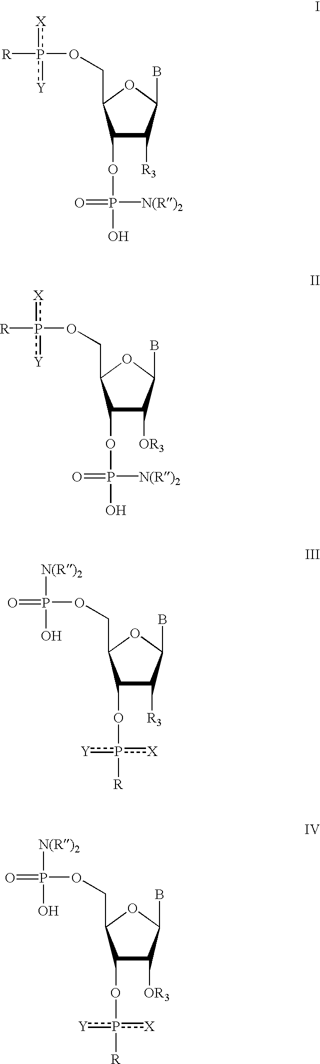
wherein in each of Formula I, II, III or IV: X and Y are each independently selected from a group consisting of O, S, Se, NR¹NR², CR¹CR², OR, SR and SeR, or one or both of X and Y are an enzymatically reactive moiety;
R is a hydrogen or non-hydrogen substituent;
each R″ is independently is hydrogen or a non-hydrogen substituent, or two R′ groups are taken together with the depicted nitrogen to form a ring;
R¹, R² and R³ are each independently selected from a group as defined by R;
B is base; and pharmaceutically acceptable salts thereof.

14. A library of claim 13 wherein R, R″, R¹, R² and R³ are each independently selected from the group of hydrogen, optionally substituted alkyl, optionally substituted alkenyl, optionally substituted alkynyl, optionally substituted aralkyl, optionally substituted cycloalkyl, optionally substituted cycloalkenyl, optionally substituted carbocyclic aryl, an optionally substituted mononucleotide, an optionally substituted polynucleotide, or an optionally substituted heteroaromatic or heteroalicyclic group preferably having from 1 to 3 separate or fused ring and 1 to 3 N, O or S atoms.

15. A library of claim 13 wherein B is optionally substituted adenine, optionally substituted thymidine, optionally substituted cytosine or an optinally substituted guanine.

16. A library of claim 13 wherein the library comprises dinucleotide compounds.

17. A library of one of claim 13 wherein the library comprises trinucleotide compounds.

18. A library of claim 13 wherein the library comprises at least about 20 distinct compounds.

19. A library of claims 13 wherein the library comprises at least about 100 distinct compounds.

20. A library of claim 13 wherein the library comprises one or more compounds of the following Formulae V and VI: 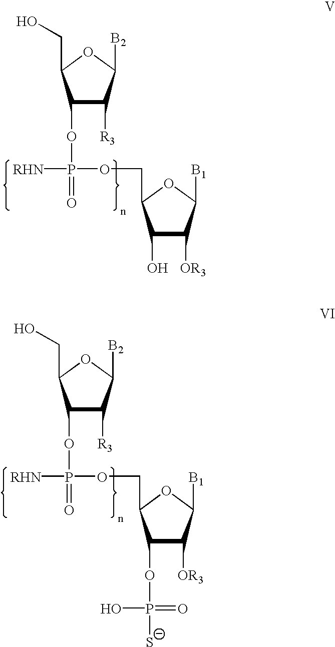
wherein in each of Formula V and VI:
each R and each R³ are each independently hydrogen or a non-hydrogen group;
B₁ and B₂ are each the same or different and are each a base; and n is a positive integer; and pharmaceutically acceptable salts thereof.

21. A method for preparing a plurality of phosphoramidate compounds, comprising contacting a plurality of nucleoside reagents with one or more amines to provide a plurality of phosphoramidate compounds.

22. The method of claim 21 wherein the nucleoside reagents are reacted with a plurality of amines.

23. The method of claim 21 wherein the nucleoside reagents are covalently bound to a solid support.

24. The method of claims 21 wherein the plurality of phosphoramidate compounds are prepared in a single reaction sequence.

25. The method of claim 21 wherein the one or more amines are independently an acyclic amine, alicyclic amine or aromatic amine.

26. The method of claim 21 the one or more amines are independently primary or secondary amines.

27. The method of claims 21 wherein at least about 20 distinct phosphoramidate compounds are provided.

28. The method of claim 21 wherein at least about 100 distinct phosphoramidate compounds are provided.

29. The method of claim 21 wherein the plurality of phosphoramidate compounds are each independently selected from the following Formulae I, II, III and IV: 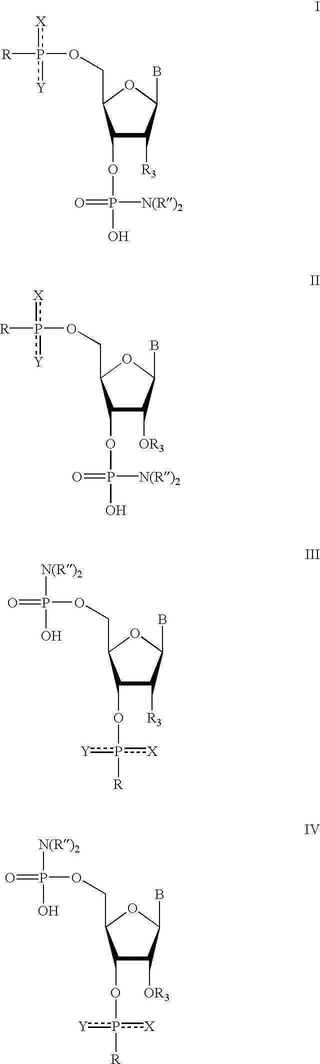
wherein in each of Formula I, II, III or IV: X and Y are each independently selected from a group consisting of O, S, Se, NR¹NR², CR¹CR², OR, SR and SeR, or one or both of X and Y are an enzymatically reactive moiety;
R is a hydrogen or non-hydrogen substituent;
each R″ is independently is hydrogen or a non-hydrogen substituent, or two R′ groups are taken together with the depicted nitrogen to form a ring;
R¹, R² and R³ are each independently selected from a group as defined by R;
B is base; and pharmaceutically acceptable salts thereof.

30. A method of claims 21 wherein the plurality of phosphoramidate compounds are each independently selected from the following Formulae V and VI: 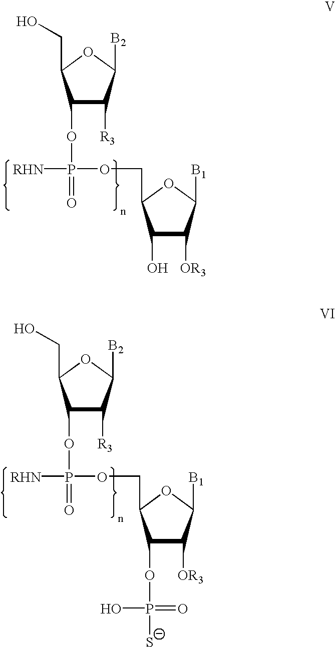
wherein in each of Formula V and VI:
each R and each R³ are each independently hydrogen or a non-hydrogen group;
B₁and B₂ are each the same or different and are each a base; and n is a positive integer; and pharmaceutically acceptable salts thereof.

31. A method of claim 30 wherein each R and each R³ are each independently selected from the group of hydrogen, optionally substituted alkyl, optionally substituted alkenyl, optionally substituted alkynyl, optionally substituted aralkyl, optionally substituted cycloalkyl, optionally substituted cyclialkenyl, optionally substituted carbocyclic aryl, an optionally substituted mononucleotide, an optionally substituted polynucleotide, or an optionally substituted heteroaromatic or heteroalicyclic group preferably having from 1 to 3 separate or fused ring and 1 to 3 N, O or S atoms.

32. A method of claim 30 wherein B₁and B₂ are each independently an optionally substituted adenine, optionally substituted thymidine, optionally substituted cytosine or an optionally substituted guanine.

33. A method of treating a subject suffering from or susceptible to a viral infection, comprising administering to the subject an effective of a compound of the following Formulae I, II, III or IV: 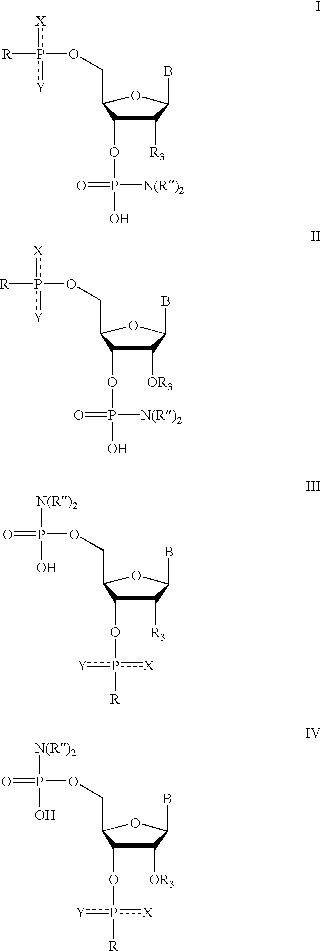
wherein in each of Formula I, II, III or IV: X and Y are each independently selected from a group consisting of O, X, Se, NR¹NR², CR¹CR², OR, SR and SeR, or one or both of X and Y are an enzymatically reactive moiety;
R is a hydrogen or non-hydrogen substituent;
each R″ is independently is hydrogen or a non-hydrogen substituent, or two R′ groups are taken together with the depicted nitrogen to form a ring;
R¹, R² and R³ are each independently selected from a group as defined by R;
B is base; and pharmaceutically acceptable salts thereof.

34. A method of claim 32 wherein the compound is of the following Formulae V or VI: 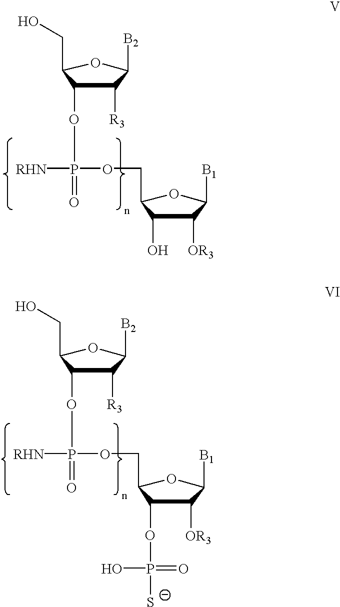
wherein in each of Formula V and VI:
each R and each R³ are each independently hydrogen or a non-hydrogen group;
B₁ and B₂ are each the same of different and are each a base; and n is a positive integer; and pharmaceutically acceptable salts thereof.

35. A method of claim 33 wherein each R and each R³ are each independently selected from the group of hydrogen, optionally substituted alkyl, optionally substituted alkenyl, optionally substituted alkynyl, optionally substituted aralkyl, optionally substituted cycloalkyl, optionally substituted cycloalkenyl, optionally substituted carbocyclic aryl, an optionally substituted mononucleotide, an optionally substituted polynucleotide, or an optionally substituted heteroaromatic or heteroalicyclic group preferably having from 1 to 3 separate or fused ring and 1 to 3 N, O or S atoms.

36. A method of claim 33 wherein B₁ and B₂ are each independently an optionally substituted adenine, optionally substituted thymidine, optionally substituted cytosine or an optionally substituted guanine.

37. A method of claim 32 wherein the subject is suffering from or susceptible to an HBV infection.
